(19) United States
(12) Patent Application Publication
(10) Pub. No.: US 2017/0209054 A1
(43) Pub. Date: Jul. 27, 2017

(54) FEEDBACK PHOTOPLETHYSMOGRAPHY MEASURING DEVICE AND MEASURING METHOD THEREOF

(71) Applicant: National Central University, Jhongli City (TW)

(72) Inventors: Po-Lei Lee, Jhongli City (TW); Kuo-Kai Shyu, Jhongli City (TW); Men-Tzung Lo, Jhongli City (TW); Hao-Teng Shu, Jhongli City (TW); Te-Min Lee, Jhongli City (TW)

(21) Appl. No.: 15/180,136

(22) Filed: Jun. 13, 2016

(30) Foreign Application Priority Data
Jan. 22, 2016 (TW) 105102054

(51) Int. Cl. A61B 5/024; A61B 5/1455; A61B 5/00

(52) U.S. Cl. CPC A61B 5/02416; A61B 5/7257; A61B 5/14551; A61B 5/02433

(57) ABSTRACT

A feedback photoplethysmography measuring device is provided, including: a measuring device that measures a human body to obtain a photoplethysmography signal; an analog/digital conversion unit that converts the photoplethysmography signal measured by the measuring device to a photoplethysmography value which is a digital value; a variation-degree calculation unit that calculates a photoplethysmography variation value according to the photoplethysmography value converted by the analog/digital conversion unit; and a feedback emitted light amplitude adjustment unit that controls the measuring device according to a plurality of preset control values or the adjusted photoplethysmography variation value calculated by the variation-degree calculation unit, and adjusts the photoplethysmography variation value to a minimum variation value.

### BACKGROUND

Technical Field

The present invention relates to the field of medical measurement, and in particular, to a feedback photoplethysmography measuring device and a measuring method thereof.

Related Art

With rapid development of industry and commerce, people's working pressure is bigger and bigger, plus an imbalanced diet, the population of cardiovascular disease is increasingly more. According to the statistics of ten leading causes of death made by the ministry of treatments of the executive yuan from 2008 to 2012, nearly half of the project is cardiovascular-related diseases, such as heart diseases, cerebrovascular diseases and hypertensive diseases. The cardiovascular-related diseases deaths accounted for 30.3% of all deaths in Taiwan in 2012, and heart diseases and cerebrovascular diseases are two leading causes of death from the cardiovascular-related diseases.

For the cardiovascular-related measurement, traditionally, a photoplethysmography measuring device is used to measure oxyhemoglobin saturation and photoplethysmography signals, which may be divided into a transmissive or reflective photoplethysmography measuring device; however, the photoplethysmography measuring device in the prior art mostly uses a light emitter that fixes brightness of light to irradiate background tissues of human bodies, and thus measured signals are often poor due to different background tissues (e.g., fingers, arms, head, stomach, legs and so on).

### SUMMARY

In view of the foregoing problems, an objective of the present invention is to provide a feedback photoplethysmography measuring device and a measuring method thereof, which can adjust brightness of light emitted by a light emitter of the photoplethysmography measuring device to be optimal with respect to different background tissues of a human body to be measured, to make the background tissues of the human body measured by the photoplethysmography measuring device have optimal measurement signals.

A first aspect of the present invention provides a feedback photoplethysmography measuring device, including:

a measuring device, including:

a light emitter that emits light with a particular wavelength to a human body; and

a light receiver that receives the light with a particular wavelength transmitted through the human body or reflected by the human body, and converts the light to a photoplethysmography signal which is an electrical signal;

an analog/digital conversion unit that converts the photoplethysmography signal converted by the light receiver which is an analog signal to a photoplethysmography value which is a digital value;

a variation-degree calculation unit that calculates a photoplethysmography variation value according to the photoplethysmography value converted by the analog/digital conversion unit; and a feedback emitted light amplitude adjustment unit that controls brightness of the light emitted by the light emitter according to a plurality of preset control values or the adjusted photoplethysmography variation value calculated by the variation-degree calculation unit, and adjusts the photoplethysmography variation value to a minimum variation value, to control the brightness of the light emitted by the light emitter to be optimal.

A second aspect of the present invention provides a feedback photoplethysmography measuring device, including:

a measuring device, including:

a plurality of light emitters that emit light with a particular wavelength but in different sequences to a human body respectively; and

a light receiver that receives the light with a particular wavelength but in different sequences transmitted through the human body or reflected by the human body, and converts the light to a plurality of photoplethysmography signals which are electrical signals;

a light emission sequence control unit that sends each of a plurality of emitted light sequence signals to each of the light emitters through phase, pulse or sine-wave control, to control time and frequency of the light emitted by each of the light emitters;

an analog/digital conversion unit that converts the photoplethysmography signals converted by the light receiver which are analog signals to a photoplethysmography value which is a digital value respectively;

a variation-degree calculation unit that calculates a plurality of photoplethysmography variation values according to the photoplethysmography values converted by the analog/digital conversion unit; and

a feedback emitted light amplitude adjustment unit that controls brightness of the light emitted by each of the light emitters according to a plurality of preset control values or the adjusted photoplethysmography variation values calculated by the variation-degree calculation unit, and adjusts at least one of the photoplethysmography variation values to a minimum variation value, to control the brightness of the light emitted by the light emitter corresponding to the minimum variation value to be optimal.

A third aspect of the present invention provides a measuring method of a feedback photoplethysmography measuring device, including:

emitting, by a light emitter, light with a particular wavelength to a human body;

receiving, by a light receiver, the light with a particular wavelength transmitted through the human body or reflected by the human body, and converting the light to a photoplethysmography signal which is an electrical signal;

converting, by an analog/digital conversion unit, the photoplethysmography signal converted by the light receiver which is an analog signal to a photoplethysmography value which is a digital value;

calculating, by a variation-degree calculation unit, a photoplethysmography variation value according to the photoplethysmography value converted by the analog/digital conversion unit; and

adjusting, by a feedback emitted light amplitude adjustment unit, the photoplethysmography variation value, and controlling brightness of the light emitted by the light emitter according to a plurality of preset control values or the adjusted photoplethysmography variation value calculated by the variation-degree calculation unit;

wherein the feedback emitted light amplitude adjustment unit judges whether the adjusted photoplethysmography variation value is the minimum variation value.

A fourth aspect of the present invention provides a measuring method of a feedback photoplethysmography measuring device, including:

emitting, by a plurality of light emitters, light with a particular wavelength but in different sequences to a human body;

receiving, by a light receiver, the light with a particular wavelength but in different sequences transmitted through the human body or reflected by the human body, and converting the light to a plurality of photoplethysmography signals which are electrical signals;

sending, by a light emission sequence control unit, each of a plurality of emitted light sequence signals to each of the light emitters through phase, pulse or sine-wave control, to control time and frequency of the light emitted by each of the light emitters;

converting, by an analog/digital conversion unit, the photoplethysmography signals converted by the light receiver which are analog signals to a plurality of a photoplethysmography value which is a digital value;

calculating, by a variation-degree calculation unit, a plurality of photoplethysmography variation values according to the photoplethysmography values converted by the analog/digital conversion unit; and

adjusting, by a feedback emitted light amplitude adjustment unit, the photoplethysmography variation values, and controlling brightness of the light emitted by each of the light emitters according to a plurality of preset control values or the adjusted photoplethysmography variation values calculated by the variation-degree calculation unit respectively;

wherein the feedback emitted light amplitude adjustment unit judges whether at least one of the adjusted photoplethysmography variation values is the minimum variation value.

### BRIEF DESCRIPTION OF THE DRAWINGS

FIG. 1 is a block diagram of a feedback photoplethysmography measuring device according to a first embodiment of the present invention;

FIG. 2A is a sequence diagram of a pulse signal of a light emission sequence control unit according to the first embodiment of the present invention;

FIG. 2B is a frequency diagram of a photoplethysmography signal of a light receiver according to the first embodiment of the present invention;

FIG. 2C is a schematic diagram of photoplethysmography variation values calculated by a variation-degree calculation unit according to the first embodiment of the present invention;

FIG. 3A is a flow chart of a method of seeking for, by a feedback emitted light amplitude adjustment unit, a minimum variation value according to the present invention;

FIG. 3B is a flow chart of another method of seeking for, by a feedback emitted light amplitude adjustment unit, a minimum variation value according to the present invention;

FIG. 4 is a flow chart of a measuring method of a feedback photoplethysmography measuring device according to the first embodiment of the present invention;

FIG. 5 is a block diagram of a feedback photoplethysmography measuring device according to a second embodiment of the present invention;

FIG. 6A is a sequence diagram of a pulse signal of a light emission sequence control unit according to the second embodiment of the present invention;

FIG. 6B is a diagram of photoplethysmography variations obtained according to different emission frequencies according to the second embodiment of the present invention;

FIG. 6C is a schematic diagram of photoplethysmography variation values calculated by a variation-degree calculation unit according to the second embodiment of the present invention;

FIG. 7 is a flow chart of a measuring method of a feedback photoplethysmography measuring device according to the second embodiment of the present invention;

FIG. 8 is a block diagram of a feedback photoplethysmography measuring device according to a third embodiment of the present invention;

FIG. 9A is a sequence diagram of a pulse signal of a light emission sequence control unit according to the third embodiment of the present invention;

FIG. 9B is a diagram of photoplethysmography variations obtained according to different emission frequencies according to the third embodiment of the present invention;

FIG. 9C is a schematic diagram of photoplethysmography variation values calculated by a variation-degree calculation unit according to the third embodiment of the present invention; and

FIG. 10 is a flow chart of a measuring method of a feedback photoplethysmography measuring device according to the third embodiment of the present invention.

### DETAILED DESCRIPTION

For the purposes of promoting an understanding of the principles of the invention, the feedback photoplethysmography measuring device and method of the present invention are described below, and in applicants' Taiwanese priority application No. 105102054, filed Jan. 22, 2016, the entire contents of which are hereby incorporated herein by reference. The written description and drawings are intended to illustrate the invention in a manner that allows persons of ordinary skill in the art to be able to understand and appreciate the broad scope of the invention and its preferred embodiments, and to enable any person skilled in the art to which the invention pertains, or with which it is most nearly connected, to make and use the same. It will nevertheless be understood that no limitation of the scope of the invention is thereby intended, with such alterations and modifications to the illustrated device being contemplated as would normally occur to one skilled in the art to which the invention relates.

### First Embodiment

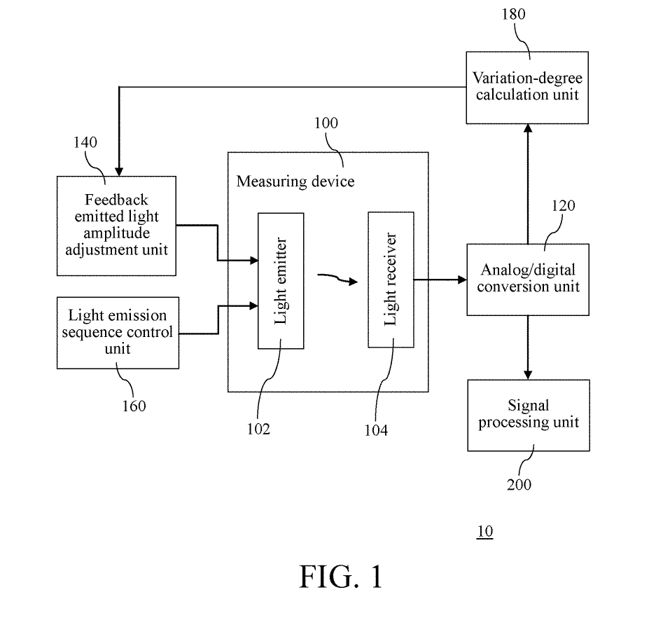

FIG. 1 is a block diagram of a feedback photoplethysmography measuring device according to a first embodiment of the present invention. In FIG. 1, the feedback photoplethysmography measuring device 10 includes a measuring device 100, an analog/digital conversion unit 120, a feedback emitted light amplitude adjustment unit 140, a light emission sequence control unit 160, a variation-degree calculation unit 180 and a signal processing unit 200. The measuring device 100 includes a light emitter 102 and a light receiver 104.

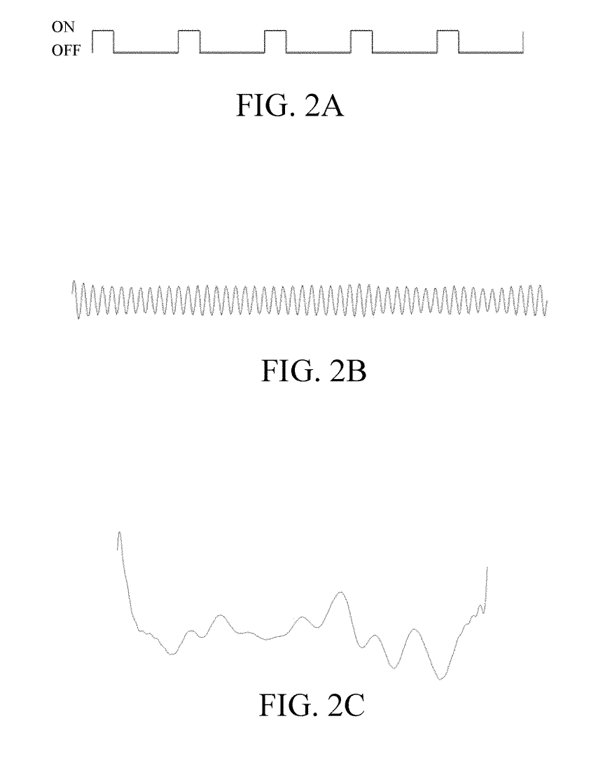

The light emission sequence control unit 160 sends an emitted light sequence signal to the light emitter 102 through phase, pulse or sine-wave control, to control time and frequency of the light emitted by the light emitter 102. As shown in FIG. 2A which is a sequence diagram of a pulse signal of a light emission sequence control unit according to the first embodiment of the present invention, the frequency of the light emitted by the light emitter 102 is the same as the frequency of the pulse signal of the light emission sequence control unit 160, and the ON cycle in the pulse signal of the light emission sequence control unit 160 makes the light emitter 102 emit light with a particular wavelength.

The light with a particular wavelength emitted by the light emitter 102 may be green light with a wavelength of 495 nm to 570 nm, red light with a wavelength of 620 nm to 750 nm or infrared light with a wavelength of 780 nm to 1000 nm.

The light receiver 104 receives light with a particular wavelength transmitted through background tissues of a human body or reflected by the background tissues of the human body, and converts the light to a photoplethysmography signal which is an electrical signal, as shown in FIG. 2B which is a frequency diagram of a photoplethysmography signal of a light receiver according to the first embodiment of the present invention.

The analog/digital conversion unit 120 converts the photoplethysmography signal converted by the light receiver which is an analog signal 104 to a photoplethysmography value which is a digital value. The variation-degree calculation unit 180 calculates a photoplethysmography variation value Var according to the photoplethysmography value converted by the analog/digital conversion unit 120.

As shown in FIG. 2C which is a schematic diagram of photoplethysmography variation values calculated by a variation-degree calculation unit according to the first embodiment of the present invention, the photoplethysmography variation value is

$$Var = \frac{1}{N} [ {\sum }_{n = 1}^{N} {( I [ n ] - M )}^{2} ] ,$$

wherein N is the total data number of the photoplethysmography value through sequence analysis or Fourier transform, I[n] is the corresponding light intensity of the photoplethysmography value via sequence analysis or Fourier transform at the time n, and M is an average value of I[n].

The feedback emitted light amplitude adjustment unit 140 controls the brightness of the light emitted by the light emitter 102 according to a plurality of preset control values or the adjusted photoplethysmography variation value Var calculated by the variation-degree calculation unit 180, and until the photoplethysmography variation value Var converted and calculated according to the photoplethysmography signal converted by the light receiver 104 is a minimum variation value, the feedback emitted light amplitude adjustment unit 140 controls the brightness of the light emitted by the light emitter 102 to be optimal.

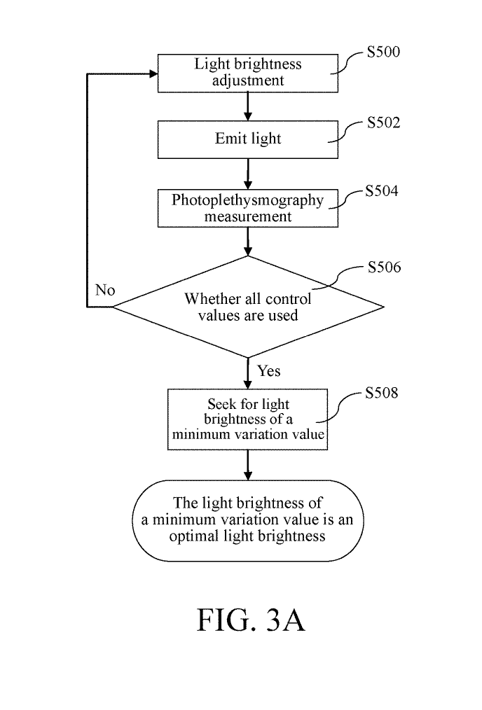

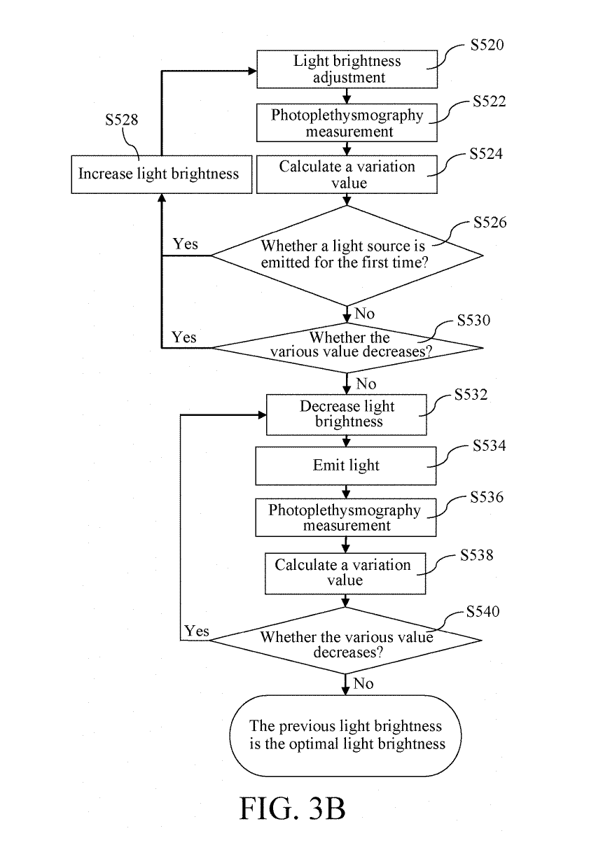

FIG. 3A is a flow chart of a method of seeking for, by a feedback emitted light amplitude adjustment unit, a minimum variation value according to the present invention. In FIG. 3A, the feedback emitted light amplitude adjustment unit 140 first adjusts the brightness of the light emitted by the light emitter 102 according to a first control value in the plurality of control values (step S500), and the light emitter 102 emits the brightness of the light corresponding to the first control value of the feedback emitted light amplitude adjustment unit 140 (step S502).

The light receiver 104 receives the light with a particular wavelength transmitted through background tissues of a human body or reflected by the background tissues of the human body, and converts the light to a photoplethysmography which is an electrical signal, the analog/digital conversion unit 120 converts the photoplethysmography signal converted by the light receiver which is an analog signal 104 to a photoplethysmography value which is a digital value, and the variation-degree calculation unit 180 calculates a photoplethysmography variation value Var according to the photoplethysmography value converted by the analog/digital conversion unit 120 (step S504).

Next, the feedback emitted light amplitude adjustment unit 140 receives the photoplethysmography variation value Var calculated by the variation-degree calculation unit 180, and judges whether all control values have been used to adjust the brightness of the light emitted by the light emitter 102 (step S506).

As the feedback emitted light amplitude adjustment unit 140 adjusts the brightness of the light emitted by the light emitter 102 with the first control value but not all the control values are used, next, step S500 is performed, to adjust the brightness of the light emitted by the light emitter 102 with a second control value, and when step S500 is performed subsequently, the brightness of the light emitted by the light emitter 102 is adjusted with a subsequent control value.

If the feedback emitted light amplitude adjustment unit 140 has used all the control values to adjust the brightness of the light emitted by the light emitter 102, the feedback emitted light amplitude adjustment unit 140 seeks for a minimum value in each photoplethysmography variation value Var corresponding to each control value, and the minimum value is the minimum variation value with which the feedback emitted light amplitude adjustment unit 140 controls the brightness of the light emitted by the light emitter 102 to be optimal (step S508).

FIG. 3B is a flow chart of another method of seeking for, by a feedback emitted light amplitude adjustment unit, a minimum variation value according to the present invention. In FIG. 3B, the feedback emitted light amplitude adjustment unit 140 first controls the brightness of the light emitted by the light emitter 102 with a preset value (step S520).

The light receiver 104 receives the light with a particular wavelength transmitted through background tissues of the human body or reflected by the background tissues of the human body, and converts the light to a photoplethysmography signal which is an electrical signal (step S522).

The analog/digital conversion unit 120 converts the photoplethysmography signal converted by the light receiver which is an analog signal 104 to a photoplethysmography value which is a digital value, and the variation-degree calculation unit 180 calculates a photoplethysmography variation value Var according to the photoplethysmography value converted by the analog/digital conversion unit 120 (step S524).

Next, the feedback emitted light amplitude adjustment unit 140 receives the photoplethysmography variation value Var calculated by the variation-degree calculation unit 180, and judges whether the light emitter 120 is controlled to emit light for the first time (step S526).

As the feedback emitted light amplitude adjustment unit 140 controls the light emitter 120 to emit light for the first time, the feedback emitted light amplitude adjustment unit 140 adjusts the size of the received photoplethysmography variation value Var to control the brightness of the light emitted by the light emitter 102 to increase (step S528), and next, step S500 is performed, to control the brightness of the light emitted by the light emitter 102 with the adjusted photoplethysmography variation value Var (step S530).

When steps S520, S522 and S524 are performed more than twice, in step S526, this is not the first time that the feedback emitted light amplitude adjustment unit 140 controls the light emitter 102 to emit light, the feedback emitted light amplitude adjustment unit 140 judges whether the current photoplethysmography variation value Var calculated by the variation-degree calculation unit 180 is less than the previous photoplethysmography variation value Var (step S530).

If the current photoplethysmography variation value Var is less than the previous photoplethysmography variation value Var, steps S520, S522, S524, S526, S528 and so on are performed; if the current photoplethysmography variation value Var is greater than the previous photoplethysmography variation value Var, the feedback emitted light amplitude adjustment unit 140 adjusts the size of the received photoplethysmography variation value Var to control the brightness of the light emitted by the light emitter 102 to decrease (step S532), and the feedback emitted light amplitude adjustment unit 140 controls the brightness of the light emitted by the light emitter 102 with the adjusted photoplethysmography variation value Var (step S534).

The light receiver 104 receives the light with a particular wavelength transmitted through background tissues of the human body or reflected by the background tissues of the human body, and converts the light to a photoplethysmography signal which is an electrical signal (step S536).

The analog/digital conversion unit 120 converts the photoplethysmography signal converted by the light receiver which is an analog signal 104 to a photoplethysmography value which is a digital value, and the variation-degree calculation unit 180 calculates a photoplethysmography variation value Var according to the photoplethysmography value converted by the analog/digital conversion unit 120 (step S538).

The feedback emitted light amplitude adjustment unit 140 judges whether the current photoplethysmography variation value Var calculated by the variation-degree calculation unit 180 is less than the previous photoplethysmography variation value Var (step S540).

If the current photoplethysmography variation value Var is less than the previous photoplethysmography variation value Var, steps S532, S534, S536, S538 and so on are performed; if the current photoplethysmography variation value Var is greater than the previous photoplethysmography variation value Var, the previous photoplethysmography variation value Var is a minimum variation value, and the feedback emitted light amplitude adjustment unit 140 controls the brightness of the light emitted by the light emitter 102 to be optimal with the minimum variation value.

The signal processing unit 200 calculates a heart rate value with the photoplethysmography value when the photoplethysmography variation value is the minimum variation value. The heart rate value=60/time difference (heart rate/minute), and the time difference is a time length between adjacent crests in the photoplethysmography value.

A measuring method of a feedback photoplethysmography measuring device according to the first embodiment of the present invention is described with reference to the block diagram of the feedback photoplethysmography measuring device according to the first embodiment of the present invention.

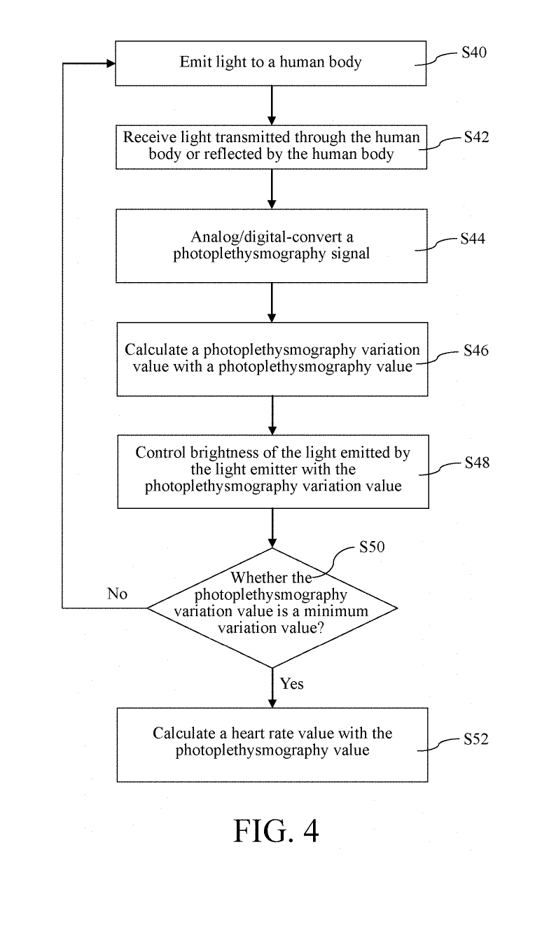

FIG. 4 is a flow chart of a measuring method of a feedback photoplethysmography measuring device according to the first embodiment of the present invention. In FIG. 3, the light emission sequence control unit 160 sends a pulse signal which is the emitted light sequence signal in FIG. 2A to the light emitter 102, to control time and frequency of the light emitted by the light emitter 102, the feedback emitted light amplitude adjustment unit 140 controls brightness of the light emitted by the light emitter 102 with a preset control value to be preset brightness, and the light emitter 102 emits the light with the preset brightness to background tissues of the human body (step S40).

The light receiver 104 receives the light with a particular wavelength transmitted through background tissues of the human body or reflected by the background tissues of the human body, and converts the light to a photoplethysmography signal which is an electrical signal as shown in FIG. 2B (step S42).

The analog/digital conversion unit 120 converts the photoplethysmography signal converted by the light receiver which is an analog signal 104 to a photoplethysmography value which is a digital value (step S44). The variation-degree calculation unit 180 calculates a photoplethysmography variation value Var according to the photoplethysmography value converted by the analog/digital conversion unit (step S46).

When the photoplethysmography variation value Var in the steps of the flow as shown in FIG. 3A or FIG. 3B is not a minimum variation value, the feedback emitted light amplitude adjustment unit 140 controls the light emitted by the light emitter 102 to be optimal with the photoplethysmography variation value Var which is the minimum variation value (step S48).

As the steps of the flow shown in FIG. 3A or FIG. 3B, the feedback emitted light amplitude adjustment unit 140 judges whether the calculated photoplethysmography variation value Var is a minimum variation value (step S50), if no, step S40 is performed; and if yes, the signal processing unit 200 calculates the heart rate value with the photoplethysmography variation value Var which is the minimum variation value (step S52).

### Second Embodiment

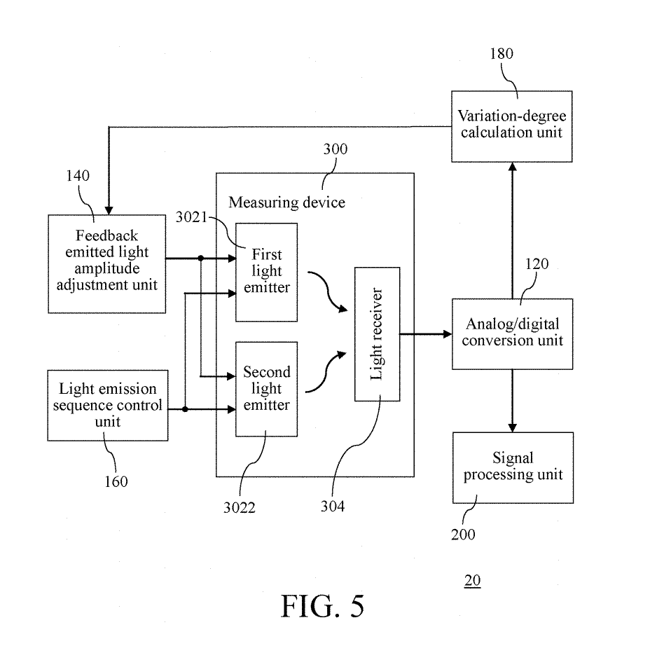

FIG. 5 is a block diagram of a feedback photoplethysmography measuring device according to a second embodiment of the present invention. In FIG. 5, the feedback photoplethysmography measuring device 20 includes a measuring device 300, an analog/digital conversion unit 120, a feedback emitted light amplitude adjustment unit 140, a light emission sequence control unit 160, a variation-degree calculation unit 180 and a signal processing unit 200. The measuring device 300 includes a first light emitter 3021, a second light emitter 3022 and a light receiver 304.

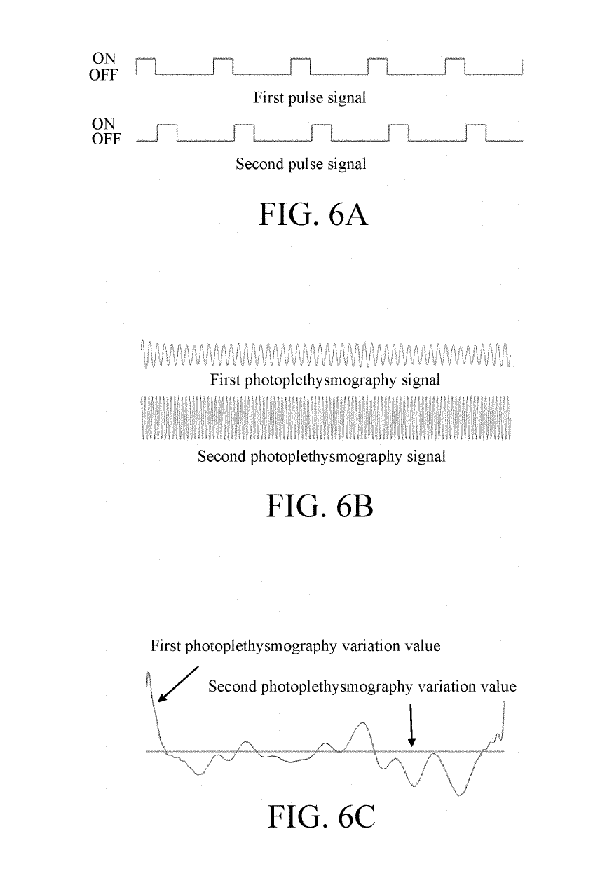

The light emission sequence control unit 160 sends two groups of pulse signals which are emitted light sequence signals to the first light emitter 3021 and the second light emitter 3022 through phase, pulse or sine-wave control, to control time and frequency of the light emitted by the first light emitter 3021 and the second light emitter 3022. As shown in FIG. 6A which is a sequence diagram of a pulse signal of a light emission sequence control unit according to the second embodiment of the present invention, the frequency of the light emitted by the first light emitter 3021 and the second light emitter 3022 is the same as the frequency of a first pulse signal and a second pulse signal of the light emission sequence control unit 160, and ON cycles in the first pulse signal and the second pulse signal of the light emission sequence control unit 160 makes the first light emitter 3021 and the second light emitter 3022 emit red light and infrared light with a particular wavelength.

The light emitted by the first light emitter 3021 may be red light with a wavelength of 620 nm to 750 nm and the light emitted by the second light emitter 3022 may be infrared light with a wavelength of 780 nm to 1000 nm.

The light receiver 304 receives red light and infrared light with a particular wavelength transmitted through background tissues of a human body or reflected by the background tissues of the human body, and converts the light to a first photoplethysmography signal and a second photoplethysmography signal which is an electrical signal, as shown in FIG. 6B which is a diagram of photoplethysmography variations obtained according to different emission frequencies according to the second embodiment of the present invention.

The analog/digital conversion unit 120 converts the first photoplethysmography signal and the second photoplethysmography signal converted to analog signals by the light receiver 104 to a first photoplethysmography value and a second photoplethysmography value which are digital values respectively. The variation-degree calculation unit 180 calculates a first photoplethysmography variation value and a second photoplethysmography variation value as the above photoplethysmography variation value Var according to the first photoplethysmography value and the second photoplethysmography value converted by the analog/digital conversion unit 120, as shown in FIG. 6C which is a schematic diagram of photoplethysmography variation values calculated by a variation-degree calculation unit according to the second embodiment of the present invention.

The feedback emitted light amplitude adjustment unit 140 controls sizes of the brightness of the light emitted by the first light emitter 3021 and the second light emitter 3022 respectively according to a plurality of preset control values or the adjusted first photoplethysmography variation value and second photoplethysmography variation value calculated by the variation-degree calculation unit 180, as the steps of the flow shown in FIG. 3A or FIG. 3B, until the first photoplethysmography variation value and the second photoplethysmography variation value converted and calculated according to the first photoplethysmography signal and the second photoplethysmography signal converted by the light receiver 304 is a minimum variation value respectively, the feedback emitted light amplitude adjustment unit 140 controls the brightness of the light emitted by the first light emitter 3021 and the second light emitter 3022 to be optimal.

The signal processing unit 200 calculates a ratio R with the first photoplethysmography value and the second photoplethysmography value when the first photoplethysmography variation value and the second photoplethysmography variation value are a minimum variation value respectively, and obtains a blood oxygen concentration value by looking up a table.

The blood oxygen concentration value is

$$R = [ \frac{( {S}_{R\_max} - {S}_{R\_min} )}{{S}_{{R}_{\_mean}}} ] / [ \frac{{S}_{I R\_max} - {S}_{I R\_min}}{{S}_{{I R}_{\_mean}}} ] ,$$

wherein S_{R}_{_}ₘₐₓ is the crest value in the photoplethysmography variation value of which the corresponding minimum variation value is red light, S_{R}_{_}ₘᵢₙ is the trough value in the photoplethysmography variation value of which the corresponding minimum variation value is red light, S_{R}_{_}ₘₑₐₙ=(S_{R}_{_}ₘₐₓ+S_{R}_{_}ₘᵢₙ)/2, S_{IR}_{_}ₘₐₓ is the crest value in the photoplethysmography variation value of which the corresponding minimum variation value is infrared light, S_{IR}_{_}ₘᵢₙ is the trough value in the photoplethysmography variation value of which the corresponding minimum variation value is infrared light, and S_{IR}_{_}ₘₑₐₙ=(S_{IR}_{_}ₘₐₓ+S_{IR}_{_}ₘᵢₙ)/2.

A measuring method of a feedback photoplethysmography measuring device according to the second embodiment of the present invention is described with reference to the block diagram of the feedback photoplethysmography measuring device according to the second embodiment of the present invention.

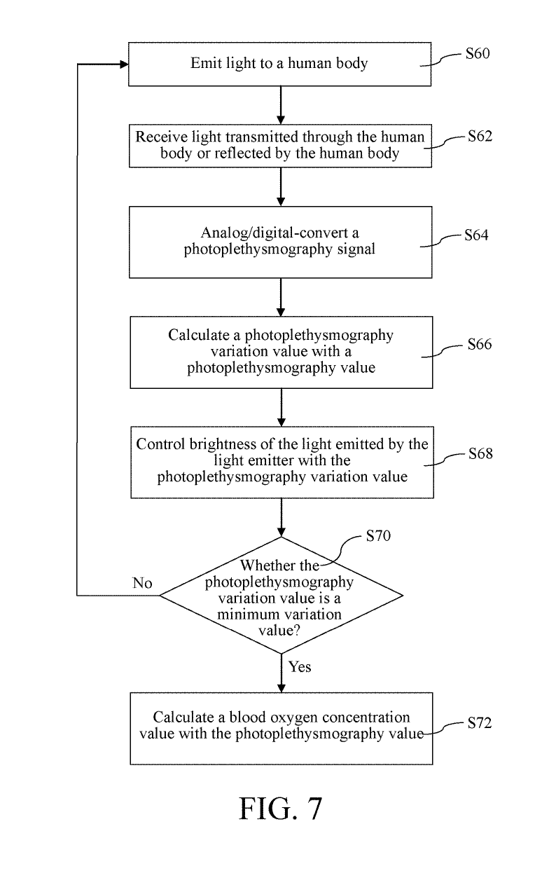

FIG. 7 is a flow chart of a measuring method of a feedback photoplethysmography measuring device according to the second embodiment of the present invention. In FIG. 7, the light emission sequence control unit 160 sends a first pulse signal and a second pulse signal which are the emitted light sequence signals in FIG. 6A to the first light emitter 3021 and the second light emitter 3022, to control time and frequency of the red light and the infrared light emitted by the first light emitter 3021 and the second light emitter 3022, the feedback emitted light amplitude adjustment unit 140 controls brightness of the light emitted by the first light emitter 3021 and the second light emitter 3022 with a first preset control value and a second preset control value to be preset brightness, and the first light emitter 3021 and the second light emitter 3022 emit the red light and the infrared light with the preset brightness to background tissues of the human body (step S60).

The light receiver 304 receives the red light and the infrared light with a particular wavelength transmitted through background tissues of the human body or reflected by the background tissues of the human body, and converts the light to a first photoplethysmography signal and a second photoplethysmography signal which is an electrical signal as shown in FIG. 6B (step S62).

The analog/digital conversion unit 120 converts the first photoplethysmography signal and the second photoplethysmography signal converted to analog signals by the light receiver 304 to a first photoplethysmography value and a second photoplethysmography value which are digital values (step S64).

The variation-degree calculation unit 180 calculates a first photoplethysmography variation value and a second photoplethysmography variation value according to the first photoplethysmography value and the second photoplethysmography value converted by the analog/digital conversion unit 120 (step S66).

When the first photoplethysmography variation value and the second photoplethysmography variation value in the steps of the flow as shown in FIG. 3A or FIG. 3B are not a minimum variation value, the feedback emitted light amplitude adjustment unit 140 controls sizes of the first photoplethysmography variation value and the second photoplethysmography variation value, and the values thereof control the sizes of brightens of the red light and the infrared light emitted by the first light emitter 3021 and the second light emitter 3022 respectively; when the first photoplethysmography variation value and the second photoplethysmography variation value are both calculated as the minimum variation value, the feedback emitted light amplitude adjustment unit 140 controls the red light and the infrared light emitted by the first light emitter 3021 and the second light emitter 3022 to be optimal with the first photoplethysmography variation value and the second photoplethysmography variation value which are the minimum variation value (step S68).

As the steps of the flow shown in FIG. 3A or FIG. 3B, the feedback emitted light amplitude adjustment unit 140 judges whether the calculated first photoplethysmography variation value and second photoplethysmography variation value are a minimum variation value (step S70), if no, step S60 is performed; and if yes, the signal processing unit 200 calculates the blood oxygen concentration value with the first photoplethysmography variation value and the second photoplethysmography variation value which are the minimum variation value (step S72).

### Third Embodiment

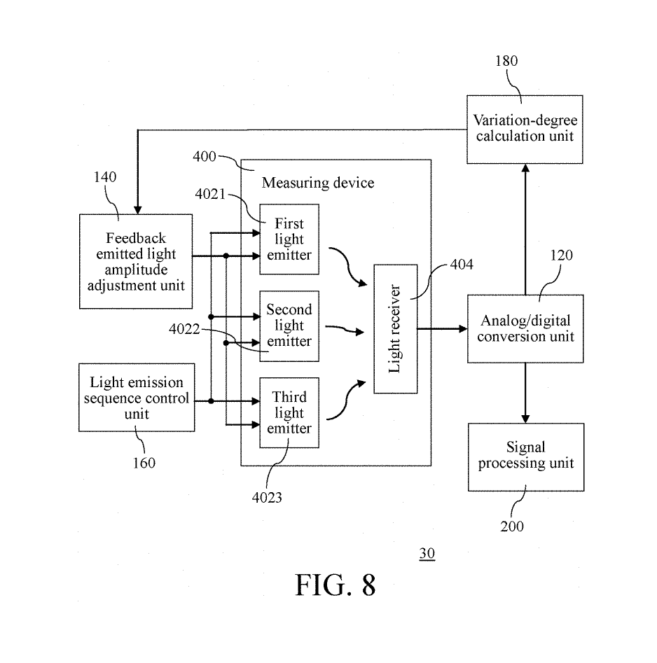

FIG. 8 is a block diagram of a feedback photoplethysmography measuring device according to a third embodiment of the present invention. In FIG. 8, the feedback photoplethysmography measuring device 300 includes a measuring device 400, an analog/digital conversion unit 120, a feedback emitted light amplitude adjustment unit 140, a light emission sequence control unit 160, a variation-degree calculation unit 180 and a signal processing unit 200. The measuring device 400 includes a first light emitter 4021, a second light emitter 4022, a third light emitter 4023 and a light receiver 404. In this embodiment, the first light emitter 4021, the second light emitter 4022 and the third light emitter 4023 are three light emitters of light with a group of particular wavelengths but at different frequencies and different brightness (green light, red light or infrared light); however, the number of the light emitters is not limited to three, more than two light emitters are applicable to the present invention, and the present invention is also applicable to multiple light emitters of light with multiple groups of particular wavelengths.

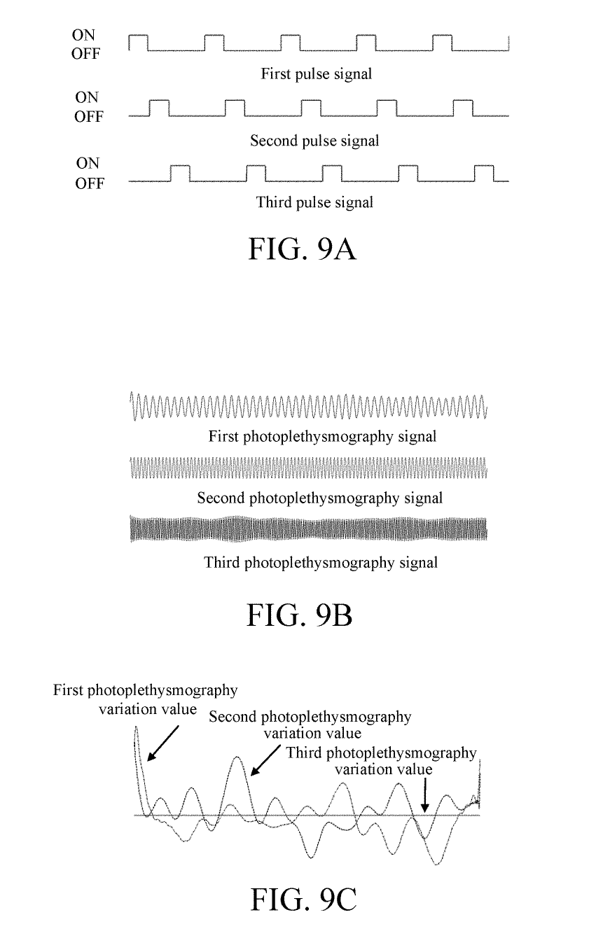

The light emission sequence control unit 160 sends three groups of pulse signals which are emitted light sequence signals to the first light emitter 4021, the second light emitter 4022 and the third light emitter 4023 through phase, pulse or sine-wave control, to control time and frequency of the light emitted by the first light emitter 4021, the second light emitter 4022 and the third light emitter 4023 respectively. As shown in FIG. 9A which is a sequence diagram of a pulse signal of a light emission sequence control unit according to the third embodiment of the present invention, the frequencies of the light emitted by the first light emitter 4021, the second light emitter 4022 and the third light emitter 4023 are the same as the frequencies of a first pulse signal, a second pulse signal and a third pulse signal of the light emission sequence control unit 160, and ON cycles in the first pulse signal, the second pulse signal and the third pulse signal of the light emission sequence control unit 160 make the first light emitter 4021, the second light emitter 4022 and the third light emitter 4023 emit light with particular wavelengths. The frequencies of the first pulse signal, the second pulse signal and the third pulse signal sent by the light emission sequence control unit 160 are different.

The light receiver 404 receives light with particular wavelengths and having different brightness and different frequencies transmitted through background tissues of a human body or reflected by the background tissues of the human body, and converts the light to a first photoplethysmography signal, a second photoplethysmography signal and a third photoplethysmography signal which are electrical signals, as shown in FIG. 9B which is a diagram of photoplethysmography variations obtained according to different emission frequencies according to the third embodiment of the present invention.

The analog/digital conversion unit 120 converts the first photoplethysmography signal, the second photoplethysmography signal and the third photoplethysmography signal converted to analog signals by the light receiver 404 to a first photoplethysmography value, a second photoplethysmography value and a third photoplethysmography value which are digital values, and the first photoplethysmography value, the second photoplethysmography value and the third photoplethysmography value respectively correspond to light with different brightness and different frequencies. The variation-degree calculation unit 180 calculates a first photoplethysmography variation value, a second photoplethysmography variation value and a third photoplethysmography variation value as the above photoplethysmography variation value Var according to the first photoplethysmography value, the second photoplethysmography value and the third photoplethysmography value converted by the analog/digital conversion unit 120, as shown in FIG. 9C which is a schematic diagram of photoplethysmography variation values calculated by a variation-degree calculation unit according to the third embodiment of the present invention.

The feedback emitted light amplitude adjustment unit 140 controls sizes of the brightness of the light emitted by the first light emitter 4021, the second light emitter 4022 and the third light emitter 4023 respectively according to a plurality of preset control values or the adjusted first photoplethysmography variation value, second photoplethysmography variation value and third photoplethysmography variation value calculated by the variation-degree calculation unit 180, as the steps of the flow shown in FIG. 3A or FIG. 3B, until one of the first photoplethysmography variation value, the second photoplethysmography variation value and the third photoplethysmography variation value converted and calculated according to the first photoplethysmography signal, the second photoplethysmography signal and the third photoplethysmography signal converted by the light receiver 104 is a minimum variation value, the feedback emitted light amplitude adjustment unit 140 controls the brightness of the light emitted by one of the first light emitter 4021, the second light emitter 4022 and the third light emitter 4023 to be optimal.

The signal processing unit 200 calculates the heart rate value with the photoplethysmography value which is the minimum variation value when one of the first photoplethysmography variation value, the second photoplethysmography variation value and the third photoplethysmography variation value is the minimum variation value. Photoplethysmography measurement is carried out with a plurality of light emitters, and when one of the plurality of light emitters is at the optimal brightness, the heart rate value is calculated with the photoplethysmography value of the light emitter which is correspondingly at the optimal brightness, in this way, the heart rate value can be quickly obtained during photoplethysmography measurement.

In another embodiment, a first group of multiple light emitters emit different-brightness and different-frequency red light with particular wavelengths, a second group of multiple light emitters emit different-brightness and different-frequency infrared light with particular wavelengths, and the signal processing unit 200 calculates the ratio R with the photoplethysmography value in the first group of multiple light emitters which is a minimum variation value and the photoplethysmography value in the second group of multiple light emitters which is a minimum variation value, and obtains a blood oxygen concentration value by looking up a table.

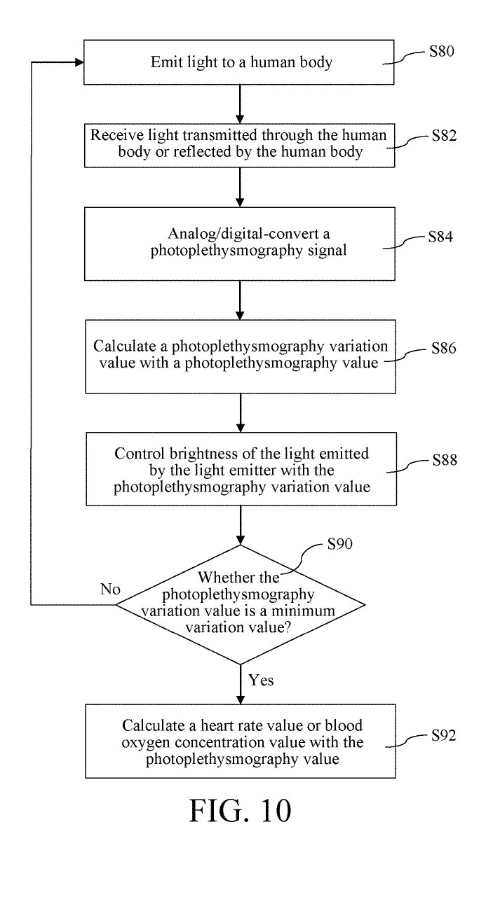

FIG. 10 is a flow chart of a measuring method of a feedback photoplethysmography measuring device according to the third embodiment of the present invention. In FIG. 10, the light emission sequence control unit 160 sends the first pulse signal, the second pulse signal and the third pulse signal in FIG. 9A which are emitted light sequence signals to the first light emitter 4021, the second light emitter 4022 and the third light emitter 4023, to control time and frequency of the light emitted by the first light emitter 4021, the second light emitter 4022 and the third light emitter 4023 respectively (the frequencies of the light emitted by the first light emitter 4021, the second light emitter 4022 and the third light emitter 4023 are different), the feedback emitted light amplitude adjustment unit 140 controls the brightness of the red light and the infrared light emitted by the first light emitter 4021, the second light emitter 4022 and the third light emitter 4023 as preset brightness with a first preset control value, a second preset control value and the third preset control value (the brightness of the light emitted by the first light emitter 4021, the second light emitter 4022 and the third light emitter 4023 is different), and the first light emitter 4021, the second light emitter 4022 and the third light emitter 4023 respectively emit light with preset brightness to background tissues of the human body (step S80).

The light receiver 404 receives a plurality of different-frequency and different-brightness light with the particular wavelengths transmitted through the background tissues of the human body or reflected by the background tissues of the human body, and converts the light to a first photoplethysmography signal, a second photoplethysmography signal and a third photoplethysmography signal which are electrical signals as shown in FIG. 9B (step S82).

The analog/digital conversion unit 120 converts the first photoplethysmography signal, the second photoplethysmography signal and the third photoplethysmography signal converted to analog signals by the light receiver 404 to a first photoplethysmography value, a second photoplethysmography value and a third photoplethysmography value which are digital values (step S84).

The variation-degree calculation unit 180 obtains a first photoplethysmography variation value, a second photoplethysmography variation value and a third photoplethysmography variation value respectively through calculation with the first photoplethysmography value, the second photoplethysmography value and the third photoplethysmography value converted by the analog/digital conversion unit 120 (step S86).

When the first photoplethysmography variation value, the second photoplethysmography variation value and the third photoplethysmography variation value in the steps of the flow as shown in FIG. 3A or FIG. 3B are not minimum variation values, the feedback emitted light amplitude adjustment unit 140 adjusts the sizes of the first photoplethysmography variation value, the second photoplethysmography variation value and the third photoplethysmography variation value, and the values thereof respectively control the sizes of brightness of the light emitted by the first light emitter 4021, the second light emitter 4022 and the third light emitter 4023, when one of the first photoplethysmography variation value, the second photoplethysmography variation value and the third photoplethysmography variation value is a minimum variation value, the feedback emitted light amplitude adjustment unit 140 controls the light emitted by the light emitter corresponding to the minimum variation value to be the optimal brightness with the photoplethysmography variation value which is the minimum variation value (step S88).

As the steps of the flow shown in FIG. 3A or FIG. 3B, the feedback emitted light amplitude adjustment unit 140 judges whether one of the calculated first photoplethysmography variation value, second photoplethysmography variation value and third photoplethysmography variation value is a minimum variation value (step S90), if no, step S80 is performed; if yes, the signal processing unit 200 calculates the heart rate value with the photoplethysmography variation value which is the minimum variation value (step S92).

In another embodiment, a first group of multiple light emitters emit different-brightness and different-frequency red light with particular wavelengths, a second group of multiple light emitters emit different-brightness and different-frequency infrared light with particular wavelengths, and the signal processing unit 200 calculates the ratio R with the photoplethysmography value in the first group of multiple light emitters which is a minimum variation value and the photoplethysmography value in the second group of multiple light emitters which is a minimum variation value, and obtains a blood oxygen concentration value by looking up a table.

The present invention provides a feedback photoplethysmography measuring device and a measuring method thereof, which can adjust brightness of light emitted by a light emitter of the photoplethysmography measuring device to be optimal with respect to different background tissues of a human body to be measured, to make the background tissues of the human body measured by the photoplethysmography measuring device have optimal measurement signals, and measure photoplethysmography with a plurality of light emitters, which can rapidly measure the heart rate value and the blood oxygen concentration value of the human body.

Although the present invention has been stated as above with reference to preferred embodiments and exemplary drawings, they should not be regarded as limitative. Various modifications, omissions and variations made by those skilled in the art to the forms and the contents of the embodiments do not depart from the scope claimed by the claims of the present invention.

What is claimed is:

1. A feedback photoplethysmography measuring device, comprising:
a measuring device, comprising:
a light emitter that emits light with a particular wavelength to a human body; and
a light receiver that receives the light with a particular wavelength transmitted through the human body or reflected by the human body, and converts the light to a photoplethysmography signal which is an electrical signal;
an analog/digital conversion unit that converts the photoplethysmography signal converted by the light receiver which is an analog signal to a photoplethysmography value which is a digital value;
a variation-degree calculation unit that calculates a photoplethysmography variation value according to the photoplethysmography value converted by the analog/digital conversion unit; and
a feedback emitted light amplitude adjustment unit that controls brightness of the light emitted by the light emitter according to a plurality of preset control values or the adjusted photoplethysmography variation value calculated by the variation-degree calculation unit, and adjusts the photoplethysmography variation value to a minimum variation value, to control the brightness of the light emitted by the light emitter to be optimal.

2. The feedback photoplethysmography measuring device according to claim 1, further comprising: a light emission sequence control unit that sends an emitted light sequence signal to the light emitter through phase, pulse or sine-wave control, to control time and frequency of the light emitted by the light emitter.

3. The feedback photoplethysmography measuring device according to claim 1, further comprising: a signal processing unit that calculates a heart rate value with the photoplethysmography value when the photoplethysmography variation value is the minimum variation value.

4. The feedback photoplethysmography measuring device according to claim 3, wherein the heart rate value=60/time difference (heart rate/minute), and the time difference is a time length between adjacent crests in the photoplethysmography value.

5. The feedback photoplethysmography measuring device according to claim 1, wherein the light emitted by the light emitter is one of green light with a wavelength of 495 nm to 570 nm, red light with a wavelength of 620 nm to 750 nm and infrared light with a wavelength of 780 nm to 1000 nm.

6. The feedback photoplethysmography measuring device according to claim 1, wherein the photoplethysmography variation value is
$$Var = \frac{1}{N} [ {\sum }_{n = 1}^{N} {( I [ n ] - M )}^{2} ] ,$$
wherein N is the total data number of the photoplethysmography value through sequence analysis or Fourier transform, I[n] is the corresponding light intensity of the photoplethysmography value via sequence analysis or Fourier transform at the time n, and M is an average value of I[n].

7. The feedback photoplethysmography measuring device according to claim 1, wherein the feedback emitted light amplitude adjustment unit controls the brightness of the light emitted by the light emitter with each of the control values, and judges that the minimum one in the photoplethysmography variation value of each of the corresponding control values calculated by the variation-degree calculation unit as the minimum variation value.

8. The feedback photoplethysmography measuring device according to claim 1, wherein, when the feedback emitted light amplitude adjustment unit judges that the current photoplethysmography variation value calculated by the variation-degree calculation unit is less than the previous photoplethysmography variation value, the feedback emitted light amplitude adjustment unit adjusts the current photoplethysmography variation value to control the brightness of the light emitted by the light emitter to increase, and when the current photoplethysmography variation value calculated by the variation-degree calculation unit is greater than the previous photoplethysmography variation value, the feedback emitted light amplitude adjustment unit adjusts the current photoplethysmography variation value to control the brightness of the light emitted by the light emitter to decrease, wherein, when the current photoplethysmography variation value calculated by the variation-degree calculation unit is judged to be less than the previous photoplethysmography variation value, the feedback emitted light amplitude adjustment unit adjusts the current photoplethysmography variation value to control the brightness of the light emitted by the light emitter to decrease, and when the current photoplethysmography variation value calculated by the variation-degree calculation unit is greater than the previous photoplethysmography variation value, the previous photoplethysmography variation value is the minimum variation value.

9. A feedback photoplethysmography measuring device, comprising:
a measuring device, comprising:
a plurality of light emitters that emit light with a particular wavelength but in different sequences to a human body respectively; and
a light receiver that receives the light with a particular wavelength but in different sequences transmitted through the human body or reflected by the human body, and converts the light to a plurality of photoplethysmography signals which are electrical signals;
a light emission sequence control unit that sends each of a plurality of emitted light sequence signals to each of the light emitters through phase, pulse or sine-wave control, to control time and frequency of the light emitted by each of the light emitters;
an analog/digital conversion unit that converts the photoplethysmography signals converted by the light receiver which are analog signals to a photoplethysmography value which is a digital value respectively;
a variation-degree calculation unit that calculates a plurality of photoplethysmography variation values according to the photoplethysmography values converted by the analog/digital conversion unit; and
a feedback emitted light amplitude adjustment unit that controls brightness of the light emitted by each of the light emitters according to a plurality of preset control values or the adjusted photoplethysmography variation values calculated by the variation-degree calculation unit, and adjusts at least one of the photoplethysmography variation values to a minimum variation value, to control the brightness of the light emitted by the light emitter corresponding to the minimum variation value to be optimal.

10. The feedback photoplethysmography measuring device according to claim 9, further comprising: a signal processing unit that calculates a heart rate value for the photoplethysmography value which is the minimum variation value when one of the photoplethysmography variation values is the minimum variation value.

11. The feedback photoplethysmography measuring device according to claim 10, wherein the heart rate value=60/time difference (heart rate/minute), and the time difference is a time length between adjacent crests in the photoplethysmography value which is the minimum variation value.

12. The feedback photoplethysmography measuring device according to claim 9, further comprising: a signal processing unit calculates that, when one of the photoplethysmography variation values of which the wavelength is red light and one of the photoplethysmography variation values of which the wavelength is infrared light are both the minimum variation value, a ratio R for the photoplethysmography variation value of which the wavelength of the minimum variation value is red light and the photoplethysmography variation value of which the wavelength is infrared light, and obtains a blood oxygen concentration value by looking up a table.

13. The feedback photoplethysmography measuring device according to claim 12, wherein the ratio is
$$R = [ \frac{( {S}_{R\_max} - {S}_{R\_min} )}{{S}_{{R}_{\_mean}}} ] / [ \frac{{S}_{I R\_max} - {S}_{I R\_min}}{{S}_{{I R}_{\_mean}}} ] ,$$
wherein S_{R}_{_}ₘₐₓ is the crest value in the photoplethysmography variation value of which the corresponding minimum variation value is red light, S_{R}_{_}ₘᵢₙ is the trough value in the photoplethysmography variation value of which the corresponding minimum variation value is red light, S_{R}_{_}ₘₑₐₙ=(S_{R}_{_}ₘₐₓ+S_{R}_{_}ₘᵢₙ)/2, S_{IR}_{_}ₘₐₓ is the crest value in the photoplethysmography variation value of which the corresponding minimum variation value is infrared light, S_{IR}_{_}ₘᵢₙ is the trough value in the photoplethysmography variation value of which the corresponding minimum variation value is infrared light, and S_{IR}_{_}ₘₑₐₙ=(S_{IR}_{_}ₘₐₓ+S_{IR}_{_}ₘᵢₙ)/2.

14. The feedback photoplethysmography measuring device according to claim 9, wherein the light emitted by the light emitters is one green light with a wavelength of 495 nm to 570 nm, red light with a wavelength of 620 nm to 750 nm and infrared light with a wavelength of 780 nm to 1000 nm.

15. The feedback photoplethysmography measuring device according to claim 9, wherein each of the photoplethysmography variation values is
$$Var = \frac{1}{N} [ {\sum }_{n = 1}^{N} {( I [ n ] - M )}^{2} ] ,$$
wherein N is the total data number of each of the photoplethysmography values through sequence analysis or Fourier transform, I[n] is the corresponding light intensity of each of the photoplethysmography values via sequence analysis or Fourier transform at the time n, and M is an average value of I[n].

16. The feedback photoplethysmography measuring device according to claim 9, wherein the feedback emitted light amplitude adjustment unit controls the brightness of the light emitted by the light emitter with each of the control values, and judges that the minimum one in the photoplethysmography variation value of each of the corresponding control values calculated by the variation-degree calculation unit as the minimum variation value.

17. The feedback photoplethysmography measuring device according to claim 9, wherein, when the feedback emitted light amplitude adjustment unit judges that the current photoplethysmography variation value calculated by the variation-degree calculation unit is less than the previous photoplethysmography variation value, the feedback emitted light amplitude adjustment unit adjusts the current photoplethysmography variation value to control the brightness of the light emitted by the light emitter to increase, and when the current photoplethysmography variation value calculated by the variation-degree calculation unit is greater than the previous photoplethysmography variation value, the feedback emitted light amplitude adjustment unit adjusts the current photoplethysmography variation value to control the brightness of the light emitted by the light emitter to decrease, wherein, when the current photoplethysmography variation value calculated by the variation-degree calculation unit is judged to be less than the previous photoplethysmography variation value, the feedback emitted light amplitude adjustment unit adjusts the current photoplethysmography variation value to control the brightness of the light emitted by the light emitter to decrease, and when the current photoplethysmography variation value calculated by the variation-degree calculation unit is greater than the previous photoplethysmography variation value, the previous photoplethysmography variation value is the minimum variation value.

18. A measuring method of a feedback photoplethysmography measuring device, comprising:
emitting, by a light emitter, light with a particular wavelength to a human body;
receiving, by a light receiver, the light with a particular wavelength transmitted through the human body or reflected by the human body, and converting the light to a photoplethysmography signal which is an electrical signal;
converting, by an analog/digital conversion unit, the photoplethysmography signal converted by the light receiver which is an analog signal to a photoplethysmography value which is a digital value;
calculating, by a variation-degree calculation unit, a photoplethysmography variation value according to the photoplethysmography value converted by the analog/digital conversion unit; and
adjusting, by a feedback emitted light amplitude adjustment unit, the photoplethysmography variation value, and controlling brightness of the light emitted by the light emitter according to a plurality of preset control values or the adjusted photoplethysmography variation value calculated by the variation-degree calculation unit;
wherein the feedback emitted light amplitude adjustment unit judges whether the adjusted photoplethysmography variation value is the minimum variation value.

19. The measuring method according to claim 18, wherein, in the step of controlling, by the feedback emitted light amplitude adjustment unit, brightness of the light emitted by the light emitter, a light emission sequence control unit that sends an emitted light sequence signal to the light emitter through phase, pulse or sine-wave control, to control time and frequency of the light emitted by the light emitter.

20. The measuring method according to claim 18, after the step of judging, by the feedback emitted light amplitude adjustment unit, the adjusted photoplethysmography variation value to be the minimum variation value, further comprising: calculating, by a signal processing unit, a heart rate value with the photoplethysmography value when the photoplethysmography variation value is the minimum variation value.

21. The measuring method according to claim 20, wherein the heart rate value=60/time difference (heart rate/minute), and the time difference is a time length between adjacent crests in the photoplethysmography value.

22. The measuring method according to claim 18, wherein the light emitted by the light emitter is one of green light with a wavelength of 495 nm to 570 nm, red light with a wavelength of 620 nm to 750 nm and infrared light with a wavelength of 780 nm to 1000 nm.

23. The measuring method according to claim 18, wherein the photoplethysmography variation value is
$$Var = \frac{1}{N} [ {\sum }_{n = 1}^{N} {( I [ n ] - M )}^{2} ] ,$$
wherein N is the total data number of the photoplethysmography value through sequence analysis or Fourier transform, I[n] is the corresponding light intensity of the photoplethysmography value via sequence analysis or Fourier transform at the time n, and M is an average value of I[n].

24. The measuring method according to claim 18, wherein the feedback emitted light amplitude adjustment unit controls the brightness of the light emitted by the light emitter with each of the control values, and judges that the minimum one in the photoplethysmography variation value of each of the corresponding control values calculated by the variation-degree calculation unit as the minimum variation value.

25. The measuring method according to claim 18, wherein, when the feedback emitted light amplitude adjustment unit judges that the current photoplethysmography variation value calculated by the variation-degree calculation unit is less than the previous photoplethysmography variation value, the feedback emitted light amplitude adjustment unit adjusts the current photoplethysmography variation value to control the brightness of the light emitted by the light emitter to increase, and when the current photoplethysmography variation value calculated by the variation-degree calculation unit is greater than the previous photoplethysmography variation value, the feedback emitted light amplitude adjustment unit adjusts the current photoplethysmography variation value to control the brightness of the light emitted by the light emitter to decrease, wherein, when the current photoplethysmography variation value calculated by the variation-degree calculation unit is judged to be less than the previous photoplethysmography variation value, the feedback emitted light amplitude adjustment unit adjusts the current photoplethysmography variation value to control the brightness of the light emitted by the light emitter to decrease, and when the current photoplethysmography variation value calculated by the variation-degree calculation unit is greater than the previous photoplethysmography variation value, the previous photoplethysmography variation value is the minimum variation value.

26. A measuring method of a feedback photoplethysmography measuring device, comprising:
emitting, by a plurality of light emitters, light with a particular wavelength but in different sequences to a human body;
receiving, by a light receiver, the light with a particular wavelength but in different sequences transmitted through the human body or reflected by the human body, and converting the light to a plurality of photoplethysmography signals which are electrical signals;
sending, by a light emission sequence control unit, each of a plurality of emitted light sequence signals to each of the light emitters through phase, pulse or sine-wave control, to control time and frequency of the light emitted by each of the light emitters;
converting, by an analog/digital conversion unit, the photoplethysmography signals converted by the light receiver which are analog signals to a plurality of a photoplethysmography value which is a digital value;
calculating, by a variation-degree calculation unit, a plurality of photoplethysmography variation values according to the photoplethysmography values converted by the analog/digital conversion unit; and
adjusting, by a feedback emitted light amplitude adjustment unit, the photoplethysmography variation values, and controlling brightness of the light emitted by each of the light emitters according to a plurality of preset control values or the adjusted photoplethysmography variation values calculated by the variation-degree calculation unit respectively;
wherein the feedback emitted light amplitude adjustment unit judges whether at least one of the adjusted photoplethysmography variation values is the minimum variation value.

27. The measuring method according to claim 26, after the step of judging, by the feedback emitted light amplitude adjustment unit, at least one of the adjusted photoplethysmography variation values to be the minimum variation value, further comprising: calculating, by a signal processing unit, a heart rate value for the photoplethysmography value which is the minimum variation value when at least one of the photoplethysmography variation values is the minimum variation value.

28. The measuring method according to claim 27, wherein the heart rate value=60/time difference (heart rate/minute), and the time difference is a time length between adjacent crests in the photoplethysmography value which is the minimum variation value.

29. The measuring method according to claim 26, after the step of judging, by the feedback emitted light amplitude adjustment unit, at least one of the adjusted photoplethysmography variation values to be the minimum variation value, further comprising: calculating, by a signal processing unit, when one of the photoplethysmography variation values of which the wavelength is red light and one of the photoplethysmography variation values of which the wavelength is infrared light are both the minimum variation value, a ratio R for the photoplethysmography variation value of which the wavelength of the minimum variation value is red light and the photoplethysmography variation value of which the wavelength is infrared light, and obtaining a blood oxygen concentration value by looking up a table.

30. The measuring method according to claim 29, wherein the ratio is
$$R = [ \frac{( {S}_{R\_max} - {S}_{R\_min} )}{{S}_{{R}_{\_mean}}} ] / [ \frac{{S}_{I R\_max} - {S}_{I R\_min}}{{S}_{{I R}_{\_mean}}} ] ,$$
wherein S_{R}_{_}ₘₐₓ is the crest value in the photoplethysmography variation value of which the corresponding minimum variation value is red light, S_{R}_{_}ₘᵢₙ is the trough value in the photoplethysmography variation value of which the corresponding minimum variation value is red light, S_{R}_{_}ₘₑₐₙ=(S_{R}_{_}ₘₐₓ+S_{R}_{_}ₘᵢₙ)/2, S_{IR}_{_}ₘₐₓ is the crest value in the photoplethysmography variation value of which the corresponding minimum variation value is infrared light, S_{IR}_{_}ₘᵢₙ is the trough value in the photoplethysmography variation value of which the corresponding minimum variation value is infrared light, and S_{IR}_{_}ₘₑₐₙ=(S_{IR}_{_}ₘₐₓ+S_{IR}_{_}ₘᵢₙ)/2.

31. The measuring method according to claim 30, wherein the light emitted by the light emitters is one green light with a wavelength of 495 nm to 570 nm, red light with a wavelength of 620 nm to 750 nm and infrared light with a wavelength of 780 nm to 1000 nm.

32. The measuring method according to claim 26, wherein each of the photoplethysmography variation values is
$$Var = \frac{1}{N} [ {\sum }_{n = 1}^{N} {( I [ n ] - M )}^{2} ] ,$$
wherein N is the total data number of each of the photoplethysmography values through sequence analysis or Fourier transform, I[n] is the corresponding light intensity of each of the photoplethysmography values via sequence analysis or Fourier transform at the time n, and M is an average value of I[n].

33. The measuring method according to claim 26, wherein the feedback emitted light amplitude adjustment unit controls the brightness of the light emitted by the light emitter with each of the control values, and judges that the minimum one in the photoplethysmography variation value of each of the corresponding control values calculated by the variation-degree calculation unit as the minimum variation value.

34. The measuring method according to claim 26, wherein, when the feedback emitted light amplitude adjustment unit judges that the current photoplethysmography variation value calculated by the variation-degree calculation unit is less than the previous photoplethysmography variation value, the feedback emitted light amplitude adjustment unit adjusts the current photoplethysmography variation value to control the brightness of the light emitted by the light emitter to increase, and when the current photoplethysmography variation value calculated by the variation-degree calculation unit is greater than the previous photoplethysmography variation value, the feedback emitted light amplitude adjustment unit adjusts the current photoplethysmography variation value to control the brightness of the light emitted by the light emitter to decrease, wherein, when the current photoplethysmography variation value calculated by the variation-degree calculation unit is judged to be less than the previous photoplethysmography variation value, the feedback emitted light amplitude adjustment unit adjusts the current photoplethysmography variation value to control the brightness of the light emitted by the light emitter to decrease, and when the current photoplethysmography variation value calculated by the variation-degree calculation unit is greater than the previous photoplethysmography variation value, the previous photoplethysmography variation value is the minimum variation value.
